(19) United States
(12) Patent Application Publication
(10) Pub. No.: US 2025/0245750 A1
(43) Pub. Date: Jul. 31, 2025

(54) SYSTEM AND METHOD FOR AN AUTOMATED HEALTHCARE INSURANCE CLAIM DENIAL APPEAL USING INTEGRATED ARTIFICIAL INTELLIGENCE AND CLINICIAN REVIEW

(71) Applicant: AUTHSNAP, INC., Cottage Grove, MN (US)

(72) Inventors: Gretchen Eileen Heinen, Cottage Grove, MN (US); Wael Khouli, Marquette, MI (US); Stacy Marie Senden Bengtson, Woodbury, MN (US); Scott Senden, Oracle, AZ (US); Zachary G. Babcock, Ostego, MN (US); Lydia Narum, Eagan, MN (US); Nicholas Maxwell Faust, Minneapolis, MN (US); Nicole Osmolski, Marquette, MI (US)

(21) Appl. No.: 19/027,927

(22) Filed: Jan. 17, 2025

Related U.S. Application Data
(60) Provisional application No. 63/624,804, filed on Jan. 25, 2024.

(51) Int. Cl. G06Q 40/08; G06N 20/00; G16H 10/60; G16H 15/00; G16H 70/20

(52) U.S. Cl. CPC G06Q 40/08; G06N 20/00; G16H 10/60; G16H 15/00; G16H 70/20

(57) ABSTRACT

Ways of processing a denied insurance claim are provided. A method involves utilizing a system server comprising various modules, including a data input module, a reactive AI module, a machine learning module, a user interface module, and a letter generation module. The method includes normalizing a denied insurance claim and patient history records, comparing claim data against a medical database, and generating appeal letters. A system incorporates clinician feedback through a continuous improvement cycle where a user actively participates in training the system by reviewing and selecting relevant aspects for specific insurance companies. The automated process helps reduce clinician fatigue and human error while maintaining clinical accuracy through the integration of trained clinicians in the review process.

### CROSS-REFERENCE TO RELATED APPLICATIONS

This application claims the benefit of U.S. Provisional Application No. 63/624,804, filed on Jan. 25, 2024. The entire disclosure of the above application is incorporated herein by reference.

### FIELD

The present technology relates to review of an insurance claim review and, more specifically, ways of using algorithmic training, reactive machine AI, and generative AI together with clinician participation in appealing a healthcare insurance claim denial.

### INTRODUCTION

This section provides background information related to the present disclosure which is not necessarily prior art.

In the healthcare landscape, a practitioner faces a complex and frustrating challenge when dealing with the insurance claim and appeal process. A healthcare provider must navigate an intricate web of varying standards, requirements, and criteria that differ not only between insurance companies but also between specific types of claims. When a practitioner makes a medical decision in an emergency room or clinical setting, the practitioner draws upon a wealth of contextual information-family history shared by relatives, previous medical records, vital signs, and various clinical factors observed in the healthcare setting. One issue lies in the disconnect between the decision-making process of the practitioner and the understanding of the insurance payer of the process. While the practitioner makes an informed medical decision based on their comprehensive understanding of the situation and of the patient, the payer lacks the same context. In this way, the disconnect results in a denied claim, requiring the practitioner to engage in a time-consuming appeal process that strains both human and financial resources.

The situation is further complicated as different payers maintain distinct sets of criteria and requirements for approving a claim. What may be acceptable documentation for one insurance company could be insufficient for another insurance company, creating a labyrinth of varying standards that a practitioner must navigate. The complexity of the process is magnified when a practitioner must address a denial and an appeal, as each payor may have unique requirements for what constitutes a successful appeal. The manual processes for handling these challenges can prove slow and prone to errors. The practitioner can expend considerable time and energy reviewing lengthy clinical best practices, state and federal regulations, and organization-specific criteria, which not only contributes to practitioner fatigue but can also result in lost revenue for the practitioner and increased out-of-pocket expenses for a patient when a claim is unsuccessfully appealed.

Accordingly, there is a continuing need for a way to effectively capture and translate the decision-making process of the practitioner, bridge the communication gap with the insurance payer, automate the review of medical documentation against varying payer criteria, and generate an appropriate appeal letter while maintaining the human oversight of a trained practitioner to improve the success rate of insurance claim and reduce the administrative burden on the practitioner.

### SUMMARY

In concordance with the instant disclosure, a system that can effectively capture and translate the decision-making process of the practitioner, bridge the communication gap with the insurance payer, automate the review of medical documentation against varying payer criteria, and generate an appropriate appeal letter while maintaining the human oversight of a trained practitioner to improve the success rate of insurance claim and reduce the administrative burden on the practitioner, has surprisingly been discovered.

The present technology includes articles of manufacture, systems, and processes that relate to an automated review of a denied insurance claim using an artificial intelligence system that employs algorithmic training, large language models, generative artificial intelligence to analyze medical history and data repositories against regulations, insurance payor requirements, and industry standards while incorporating practitioner feedback to generate and improve an appeal letter. The present technology includes ways to combine data input and normalization capabilities, disease state categorizations, and continuous machine learning improvement through user review to process denied claims and generate a customized appeal letter for a specific insurance company.

In certain embodiments, a method for processing a denied insurance claim includes providing a system server, the denied insurance claim, and a patient history record. The system server can include a processor and a memory on which a plurality of modules including tangible, non-transitory, processor executable instructions are stored. The plurality of modules can include a data input module, a reactive AI module, a machine learning module, a user interface module, and a letter generation module. The data input module can have a medical database for storing disease state categorizations, regulations, insurance payor requirements, and industry standards. The data input module can be in communication with the reactive AI module. The user interface module can be configured to generate a user interface that permits interaction with the system by a user. The user interface module can be in communication with the letter generation module and the machine learning module. The letter generation module can be configured to generate a first appeal letter and a second appeal letter. The letter generating module can be configured to allow the user to interact with the first appeal letter and the second appeal letter via the user interface. The letter generation module can be in communication with the reactive AI module and the machine learning module. The method can include normalizing, by the data input module, the denied insurance claim and the patient history record by scrubbing for claim data related to the denied insurance claim and the patient history record. The reactive AI module can compare the claim data to the medical database and generate a letter recommendation based on the medical database of the data input module. The method can include generating, by the letter generation module, the first appeal letter based on the claim data, the medical database, and the letter recommendation and presenting the first appeal letter, via the user interface, to the user for review. The user can review the first appeal letter to determine an editing parameter based on an insurance company. The method can include storing the editing parameter made by the user to the first appeal letter in the machine learning module. The machine learning module can update based on the editing parameter made by the user to the first appeal letter and the letter generation module can generate the second appeal letter based on the update to the machine learning module.

In certain embodiments, a system for processing a denied insurance claim in view of a patient history record includes a system server. The system server can include a processor and a memory on which a plurality of modules including tangible, non-transitory, processor executable instructions are stored. The plurality of modules can include a data input module, a reactive AI module, a machine learning module, a user interface module, and a letter generation module. The data input module can have a medical database for storing disease state categorizations, regulations, insurance payor requirements, and industry standards.

The data input module can be in communication with the reactive AI module. The user interface module can be configured to generate a user interface that permits interaction with the system server by a user. The user interface module can be in communication with the letter generation module and the machine learning module. The letter generation module can be configured to generate a first appeal letter and a second appeal letter. The letter generating module can be configured to allow the user to interact with the first appeal letter and the second appeal letter via the user interface. The letter generation module can be in communication with the reactive AI module and the machine learning module. The system server can be configured to normalize, by the data input module, the denied insurance claim and the patient history record by scrubbing for claim data related to the denied insurance claim and the patient history record. The system server can compare the claim data against the medical database and generate a letter recommendation based on the medical database of the data input module. The system server can generate a first appeal letter and present the first appeal letter to the user for review. The system server can store an editing parameter made by the user and update the machine learning module based on the editing parameter. The system server can generate a second appeal letter based on the update to the machine learning module.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is a schematic of a system for processing a denied insurance claim with a patient history record, according to one embodiment of the present disclosure;

FIGS. 2A-2C provide a flow chart depicting a method for processing a denied insurance claim, according to one embodiment of the present disclosure;

FIGS. 3A-3E provide a flow chart depicting a method for processing a denied insurance claim, according to another embodiment of the present disclosure;

FIG. 4 depicts a user interface for a user listing claim denial in the system, according to one embodiment of the present disclosure;

FIG. 5 depicts a user interface for a user providing relevant clinical information in the system, according to one embodiment of the present disclosure; and

FIG. 6 depicts an exemplary embodiment of a user interface for a user providing a first appeal letter, according to one embodiment of the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

As used herein, the term “user” or “practitioner” refers to an individual or professional who is involved in evaluating, analyzing, and disputing the denial of an insurance claim via the system. As examples, the “user” or “practitioner” can include a doctor (including a general practitioner, specialist, and surgeon), a nurse (including a registered nurse, nurse practitioner, and nurse anesthetist), a technician (including a radiologic technologist, lab technician, and respiratory therapist), a dentist, a pharmacist, and a physical therapist, a medical assistant, a dietitian, a case manager, a social worker, a speech-language pathologist, an occupational therapist, a clinical psychologist, a psychiatrist, a chiropractor, an optometrist, and a podiatrist, a healthcare administrator, a patient advocate, a healthcare attorney, a laboratory scientist, a veterinarian, a medical researcher, and a phlebotomist. A skilled artisan can select a suitable “user” or “practitioner” within the scope of the present disclosure.

As used herein, the term “payor” refers to an entity or organization responsible for paying or reimbursing the benefits outlined in an insurance policy after a claim is filed. For example, the payor can include an insurance company, government program (such as Medicare or Medicaid), or employer-sponsored health plan that processes and settles a claim on behalf of the insured party. The payor evaluates the claim based on the terms of the insurance policy, determines the amount of payment, and can either approve or deny the claim based on eligibility, coverage, and other policy criteria.

The present technology includes ways to process a denied insurance claim and review a denied insurance claim that employs a combination of algorithmic training, large language models, and generative artificial intelligence (AI) working together. As an example, the algorithmic training can include the iterative feedback loop where the system 100 captures changes to variable weights through a Change Data Capture (CDC) during the training process, as described here. As an example, the large language model can include using specialized language prompts and IP-defined definitions to generate appeal letters. As an example, the reactive machine AI can include analyzing clinical data and provides recommendations about possibly related medical conditions based on the medical database.

A method includes data input, where the system intakes substantial amounts of patient data from various file types and uses integrated Application Programming Interface (API) connections to Electronic Health Record software. The method utilizes a cycle where a user, such as a practitioner, actively participates in training the system by reviewing and selecting a relevant aspect for an insurance company. A system learns which criteria are successful in appealing an insurance coverage denial for the insurance company by tracking the response and outcome specific to the insurance company. The system utilizes a large language model to draft an appeal letter that can be edited as needed. The system specifically tracks the successful response that overcomes the claim denial by the insurance company and uses the information to improve future submissions through a continuous feedback and improvement loop where the practitioner reviews the generated response and selects the most relevant aspects for the insurance company.

The method can operably automate and streamline the process of reviewing and appealing a denied insurance claim through an integrated system that combines artificial intelligence technology with clinical expertise. The method specifically addresses challenges with prior authorization, medical necessity review, an appeal, and denial management by automating the time-consuming manual process while maintaining clinical accuracy through the integration of trained users in the review and training process.

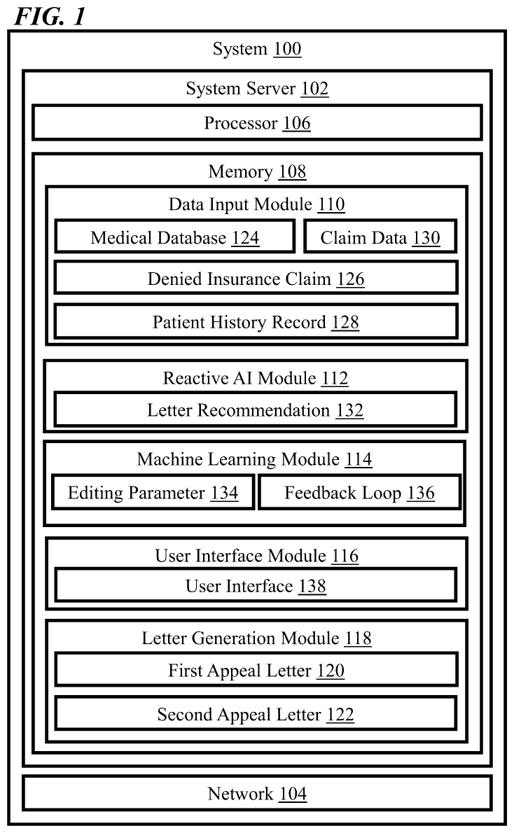

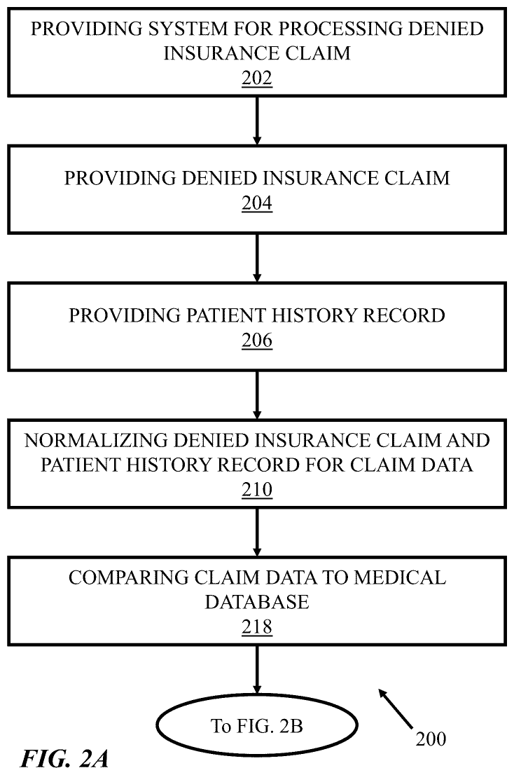

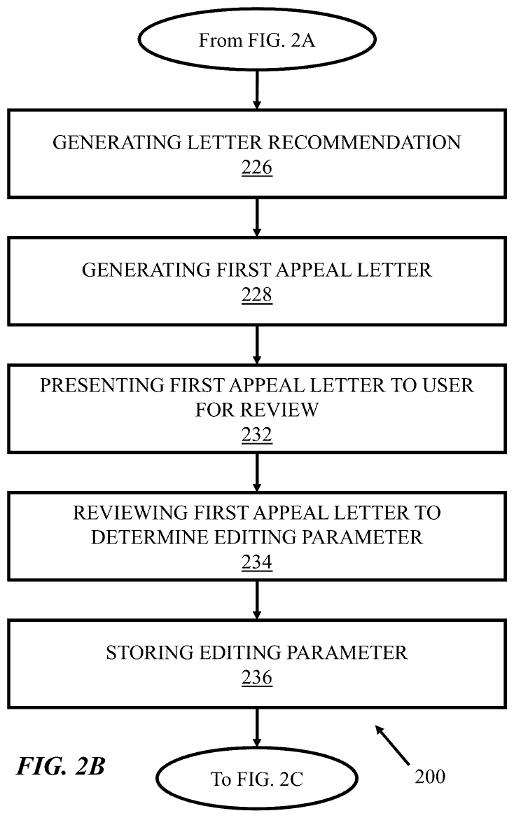

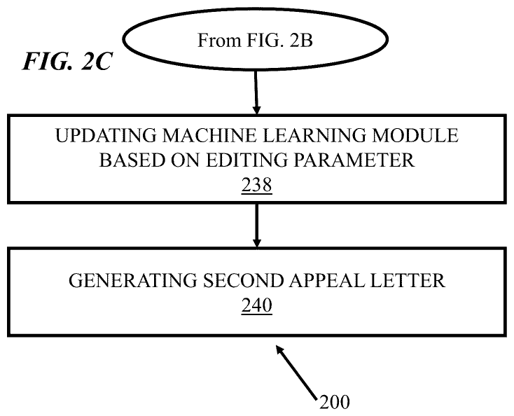

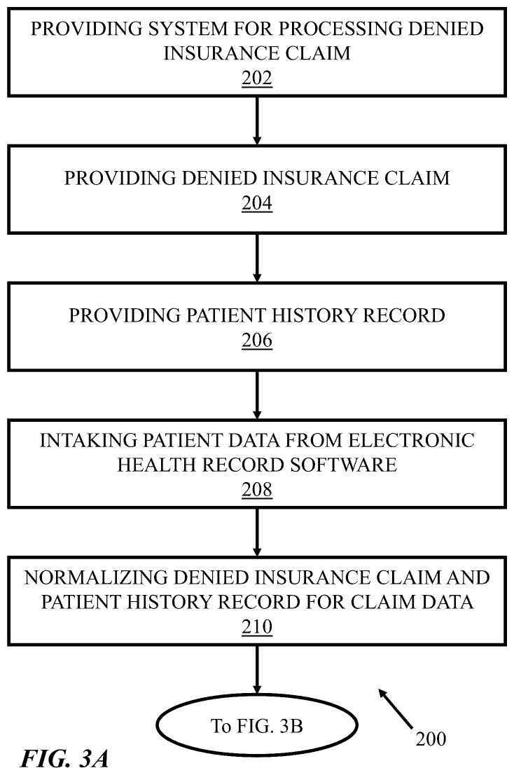

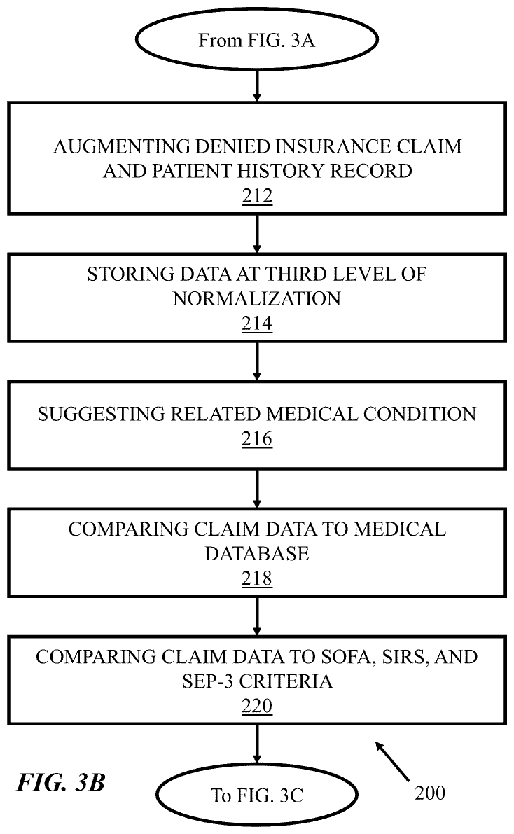

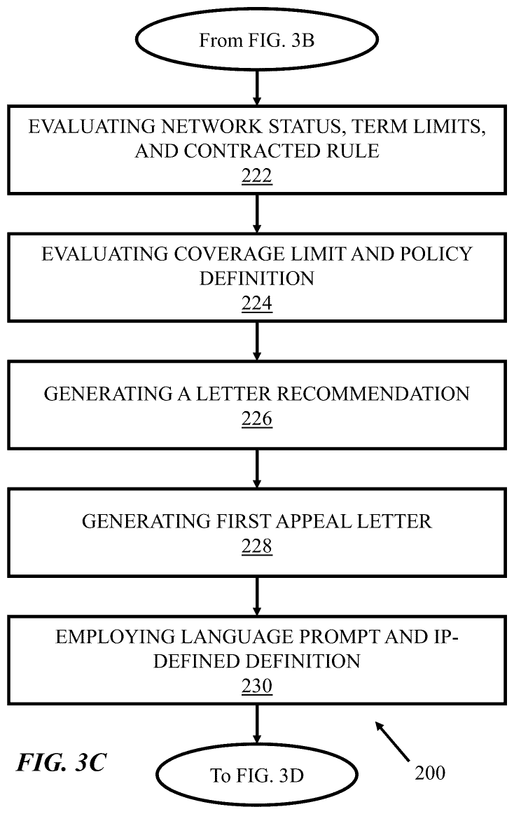

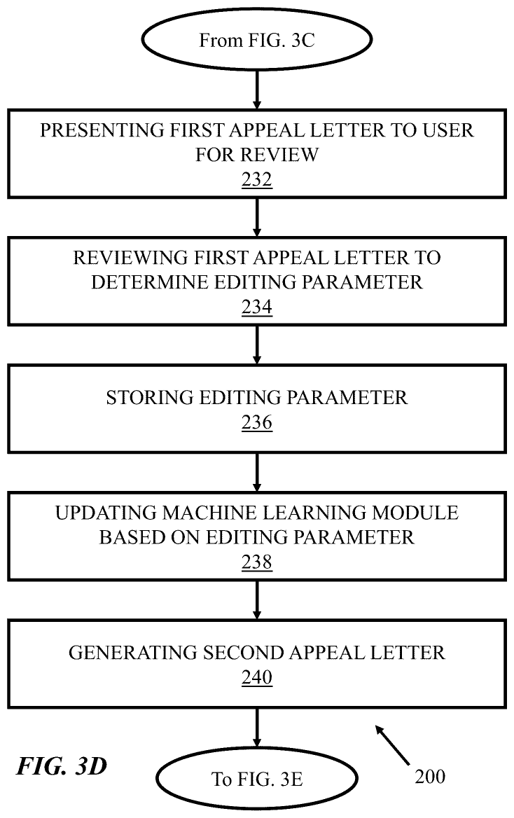

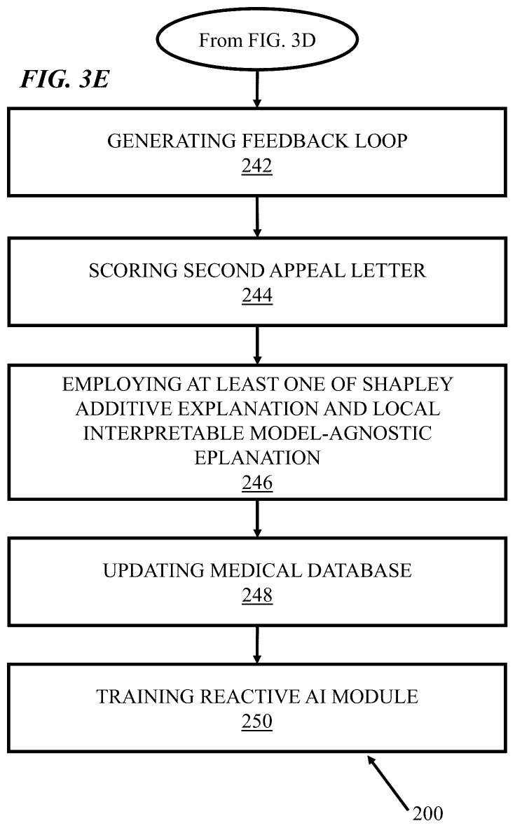

With reference now to the drawings enclosed herewith, ways to effectively capture and translate the decision-making process of the practitioner, bridge the communication gap with the insurance payer, automate the review of medical documentation against varying payer criteria, and generate an appropriate appeal letter while maintaining the human oversight of a trained practitioner to improve the success rate of insurance claim and reduce the administrative burden on the practitioner are provided. As shown in FIG. 1, a system 100 is illustrated that is configured for processing a denied insurance claim with a patient history record. For example, the denied insurance claim can be reviewed and challenged in a method 200 as shown in FIGS. 2A-2C and 3A-3E and in accordance with one or more embodiments.

In certain embodiments, as shown in FIG. 1, the system 100 can include a computing platform in the form of a system server 102. The system server 102 can be communicably coupled with a plurality of platforms, for example, via a network 104. In some cases, users can access the system 100 via the plurality of remote platforms. It should be appreciated that, depending on the situation, the system server 102 can therefore be provided as either a standalone system or a distributed system with the operable components and related operations distributed across more than one platform.

The computing platform can be communicatively coupled to the remote platform. The communicative coupling can include communicative coupling through a networked environment such as the network 104. The networked environment can be a radio access network, such as LTE or 5G, a local area network (LAN), a wide area network (WAN) such as the Internet, or wireless LAN (WLAN), for example. The computing platform can be configured to communicate with the network 104 via a wireless or wired connection. In addition, in another embodiment, the system server 102 can also include one or more hosts or servers, such as the system server 102 connected to the network 104 through wireless or wired connections. The system server 102 can be implemented in or function as base stations (which can also be referred to as Node Bs or evolved Node Bs (eNBs)). The system server 102 can include web servers, mail servers, application servers, etc. The system server 102 can be standalone servers, networked servers, or an array of servers. The plurality of remote platforms can be configured to communicate directly with each other via wireless or wired connections. Examples of the plurality of remote platforms can include smartphones, wearable devices, tablets, laptop computers, desktop computers, Internet of Things (IoT) devices, or other mobile or stationary devices.

Referring again to FIG. 1, the system 100 can be configured by machine readable instruction. The machine-readable instruction can include modules. In this respect, the method 200 as shown in FIGS. 2A-2C and 3A-3E, can be configured to be implemented by the modules, which in turn can be implemented as one or more of functional logic, hardware logic, electronic circuitry, software modules, and the like.

The system 100 can include a system server 102 having a processor 106 and memory 108. The memory 108 can store a plurality of modules including tangible, non-transitory, processor executable instructions. With reference to FIG. 1, the plurality of modules can include a data input module 110, a reactive AI module 112, a machine learning module 114, a user interface module 116, and a letter generation module 118. The modules can collaborate to generate a first appeal letter 120 and a revised second appeal letter 122 for challenging a denied insurance claim 126. It should be noted that the system 100 can generate any number of subsequent appeal letters after the second appeal letter 122 based on feedback to generate a successful appeal letter that overcomes the denial of the insurance claim, and the insurance claim is approved by the insurance company.

The data input module 110 can facilitate data processing and management tasks. The data input module 110 can process multiple file types and can integrate with Electronic Health Record systems through various API connections including REST API, GraphQL API, Web socket API, OAUTH API, Open ID connect API and SOAP API. When intaking content, the data input module 110 can store a medical database 124, as described herein, a denied insurance claim 126, and a patient history record 128.

The data input module 110 can include the medical database 124. The medical database 124 can store information relevant to the claim review process and serve as a repository that stores multiple categories of healthcare-related information, including disease state categorizations, regulations, insurance payor requirements, and industry standards, as examples, that are required for processing denied claims. As an example, a disease state categorization can include evaluating a cancer operation case where the system 100 categorizes and tracks specific parameters including the cancer operation code, cancer operation name, cancer operation plan name, cancer operation plan type, cancer diagnosis code, cancer operation coinsurance amount, cancer operation procedure approved amount, cancer operation procedure charge amount, co-payment amount, deductible amount, and noncovered charges. As an example, a regulation can include a regulation determined by a state medical board or a requirement for prior authorization from insurance companies for certain medical treatments, use of diagnostic equipment, and other procedures corresponding to an order created by a physician/provider.

As an example, an insurance payor requirement can include the need for prior authorization for a specific medical treatment, diagnostic equipment usage, and a procedure ordered by a practitioner. A payor can also have specific requirements about properly filled out payor-specific prior authorization requests, including requirements for particular fields of data and supporting documents. For instance, for a knee replacement surgery, a payor can require clinical information including specific diagnostic images showing the abnormality, diagnostic image reports, documentation of the condition requiring the procedure, severity of pain details, functional disabilities interfering with daily activities, a treatment plan including pre-op discussion, physical examination records of the relevant joint, co-morbid medical conditions, and documentation of failed therapies including dates. Additionally, payors may have specific requirements about procedure codes, diagnosis codes, and other information that must be submitted in the correct format, with some payors allowing electronic submission while others require paper submission.

As an example, an industry standard can include medical best practices and evidence-based resources when generating appeals, as determined by a medical regulation body. For instance, when evaluating a knee replacement surgery case, industry standards can include reviewing diagnostic imaging results, documentation of the condition severity, functional disabilities, treatment plan, physical examination records, and documentation of failed therapies. The system maintains these industry standards by continuously updating its medical database as clinical and society guidelines evolve to reflect current best practices. A skilled artisan can select other suitable healthcare related information for the medical database 124 to store. The medical database 124 can utilize a payer-disease state-decision structure categorization system that considers factors such as payer status (in-network, out-of-network), disease classifications, denial reasons, and patient records to assist the system 100 with generating the first appeal letter 120 and the second appeal letter 122, while continuously updating the categorization around disease states based on clinical and society guidelines stored in the medical database 124.

The medical database 124 can include comprehensive clinical information such as clinical best practices, medical guidelines, state and federal regulations, insurance payor-specific requirements and policies, medical diagnosis assessments such as Sequential Organ Failure Assessment (SOFA), Systemic Inflammatory Response Syndrome (SIRS), and Sepsis-3 (SEP-3) criteria, medical agreed terms limits, contracted rules, coverage limits, and policy definitions specific to public or private health insurance, as examples. The medical database 124 can be built and maintained through scanning public websites of payers, for example, to find documentation that defines a list of codes requiring prior authorization, as well as through partner relationships where payer data is transmitted to the system periodically.

It should be appreciated that the data input module 110 can store the medical database 124 for use and comparison with the denied insurance claim 126 and the patient history record 128 for any patient. While the medical database 124 can store more general information, the denied insurance claim 126 and the patient history record 128 can be tailored to a specific patient and the denied insurance claim 126 of the patient specific to a medical event.

It should be noted that the patient history record 128 can include the medical condition and medical background of the patient. The patient history record 128 can also include family medical history and longitudinal patient data, along with clinical information such as family history provided by family members to doctors, previous medical records, vital signs data, and clinical factors observed in an ambulatory setting. The patient history record 128 can allow the system 100 to capture the context that the practitioner uses in the decision-making and diagnosis process.

The data input module 110 can normalize the denied insurance claim 126 and patient history record 128 through data processing. The normalization process can include augmenting the data using clinical definitions. When processing the data, the data input module 110 can import, store, and scrub for patient data, storing the normalized data at a third or fourth level of normalization in the memory 104. The normalization process can include converting scanned documents and electronic documents to machine readable format using optical character recognition when necessary. The scrubbing process can produce claim data 130 that contains all information needed for the reactive AI module 112 to perform the analytical function, including evaluating the claim data 130 against disease state categorizations, regulations, insurance payor requirements, and industry standards stored in the medical database 124. The data input module 110 can curate the most relevant information pertinent to the denied insurance claim 126 in the claim data 130.

Turning now to the reactive AI module 112, it should be appreciated that the reactive AI module 112 works in concert with other modules, particularly the machine learning module 114 and letter generation module 118. The reactive AI module 112 can compare the claim data 130 against the medical database 124 to evaluate the claim data 130 for various criteria including SOFA, SIRS and SEP-3 criteria, network status including in-network status, out-of-network status, medical agreed terms limits, contracted rules, coverage limits, and policy definitions specific to the insurance company, as described herein. The reactive AI module 112 can make further suggestions to the system 100 for other medical conditions related to the denied insurance claim 126 based on the medical database 124. Upon analyzing the claim data 130, the reactive AI module 112 can generate a letter recommendation 132. The letter recommendation 132 can include a suggestion on information to include in the first appeal letter 120 based on the clinical definitions, feedback previously provided by the machine learning module 114, and other definitions of known sicknesses and medically defined vital readings known in the healthcare industry, as well as the clinical experience provided by a practitioner.

The reactive AI module 112 can incorporate the feedback provided by the machine learning module 114 during a training process where a clinician actively participates with the system 100 by reviewing and selecting relevant aspects for a specific insurance company. For example, Insurance Company A may require a specific SOFA (Sequential Organ Failure Assessment) score and SIRS (Systemic Inflammatory Response Syndrome) criteria to approve a claim for sepsis treatment. The practitioner, through experience reviewing formal denial letters, can know that Insurance Company A places more weight on these specific clinical measurements and can select the criteria for an appeal letter to Insurance Company A. In contrast, Insurance Company B may focus more on SEP-3 criteria and require additional documentation of family history and longitudinal patient data to approve the same type of claim. Through the continuous improvement cycle of the machine learning module 114, the system 100 can learn over time to incorporate company specific criteria and the reactive AI module 112 can learn from the feedback of the machine learning module 114 to include the pertinent information in the letter recommendation 132.

The machine learning module 114 can process training data and implement a feedback loop 136 to continuously improve the performance of the system 100. The machine learning module 114 can process training data, test functions to establish quantitative relationships between inputs and outputs, evaluate effects of input features on predicted outputs, tune and refine the models, and generate a quantitative relationship that the reactive AI module 112 can utilize in the letter recommendation 132.

The machine learning module 114 can utilize both supervised and unsupervised training with labeling, using the feedback loop 136 to improve letter generation by varying model weights based on clinician review. When a practitioner reviews the first appeal letter 120 with an editing parameter 134, such as correcting an error in a prediction or overriding what is produced by the system 100, the machine learning module 114 can record the editing parameter 134 and use the editing parameter 134 to improve the second appeal letter 122 and any subsequent appeal letter for the denied insurance claim 126. The machine learning module 114 can utilize a Change Data Capture (CDC) to make changes to variable weights in algorithmic training through iterative human intervention. For the purpose of the CDC, the machine learning module 114 can use explainability techniques like SHapley Additive explanations (SHAP) and Local Interpretable Model-agnostic Explanations (LIME) to provide insight into why an appeal letter was generated in a particular way.

The machine learning module 114 can store the editing parameter 134 made for each denied insurance claim 126 by the user to the appeal letters 120, 122 and incorporate the editing parameter 134 to improve future letter generation. The machine learning module 114 can track whether an appeal letter is successful in appealing the denied insurance claim 126 such that the denied insurance claim 126 is covered by the payer and use the editing parameter 134 in the feedback loop 136 for continuous improvement cycle, allowing the system 100 to become more effective over time at handling appeals and prior authorizations.

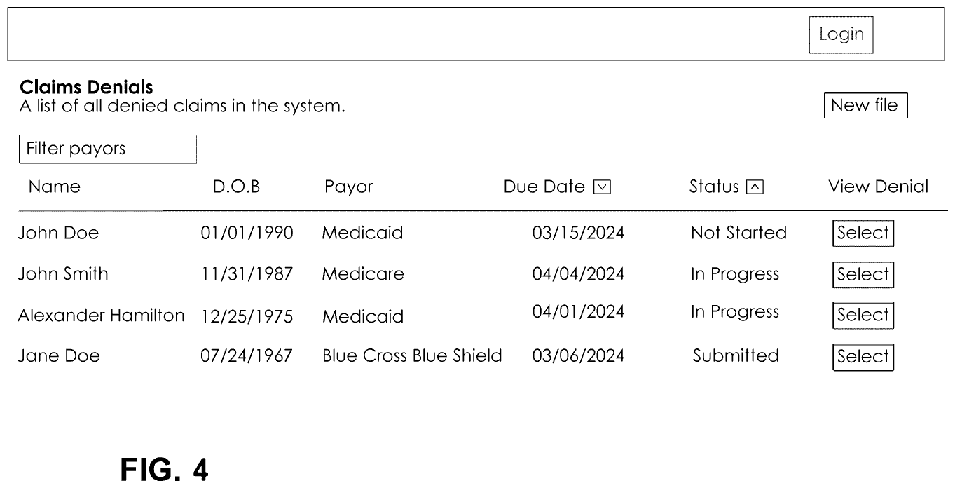

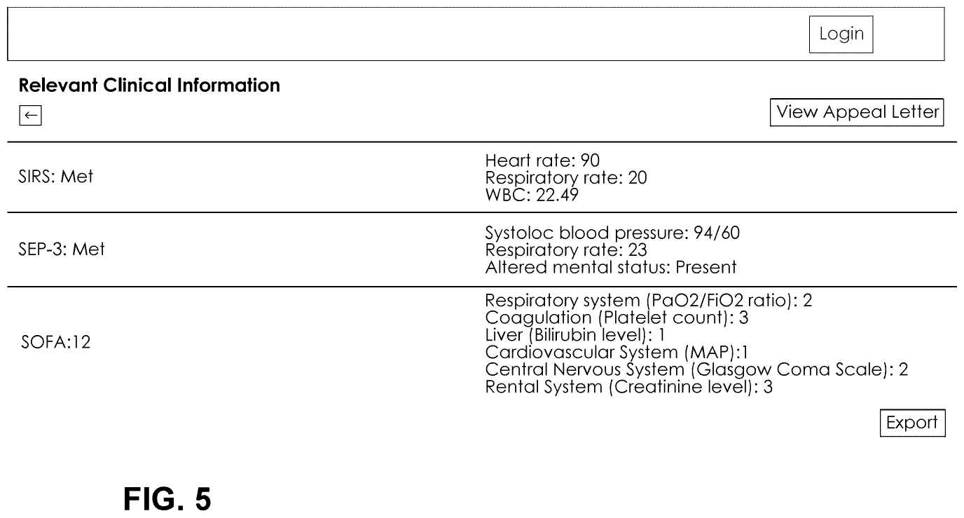

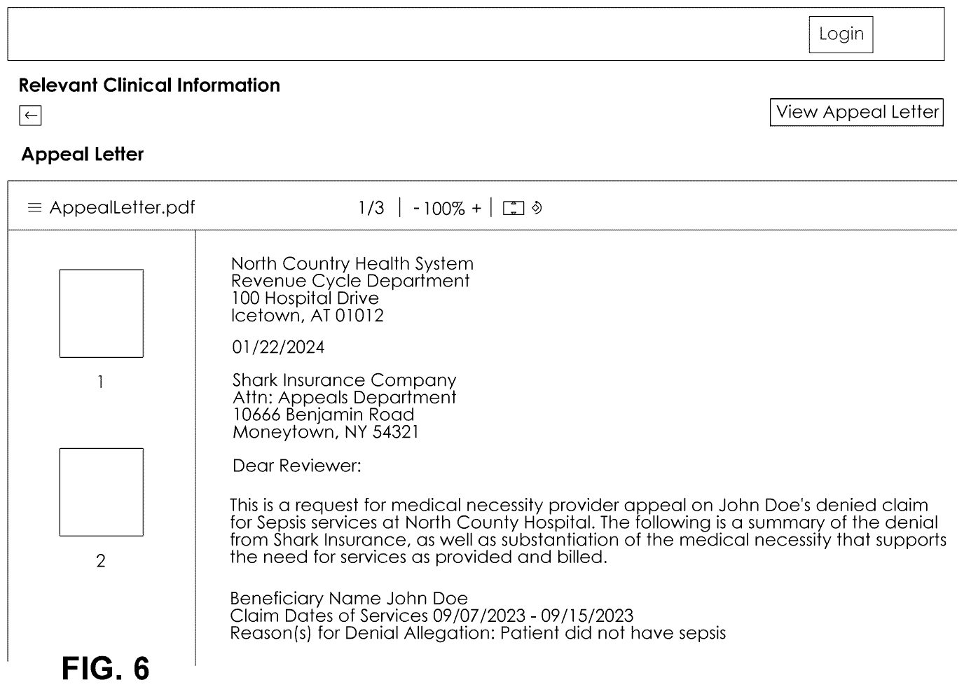

With reference to FIGS. 4-6, the user interface module 116 can allow the user to interact with the system server 102 via a user interface 138. The user interface module 116 can generate the user interface 138 to enable the user to interact with the system 100 while providing capabilities for practitioner review and document editing. Through the user interface 138, the user can review the generated first appeal letter 120 and the second appeal letter 122, select relevant aspects specific to the insurance company, and make necessary edits and changes to the appeal letters 120, 122. The user interface 138 can include interactive features such as base information input fields, denial reason documentation, and user response options through a drop-down menu and multi-select capabilities, along with fields for inputting relevant clinical criteria, as shown in FIGS. 4-6.

The user interface module 116 to be in direct communication with the machine learning module 114, allowing for the storage and processing of user edits and feedback while enabling tracking and incorporation of successful responses. The user interface module 116 can also be in communication with the letter generation module 118. The user interface module 116 can receive and display the generated appeal letters 120, 122 and permit interaction with both the first appeal letter 120 and the second appeal letter 122 via the user interface 138, allowing the user to actively participate in the training of the system 100 through letter editing. The user interface module 116 can be in communication with the reactive AI module 112, enabling the user to review and validate the letter recommendation 132 and incorporate the expertise of the user into the editing parameter 134 and ultimately, the machine learning module 114. The interconnectivity of the user interface module 116 with the other modules of the system 100 allows for the user interface module 116 to serve as a hub for managing review workflow, tracking the progress of the denied insurance claim 126 through the system 100, and tracking the overall performance of the system 100.

The letter generation module 118 can create and manage the first appeal letter 120 and the second appeal letter 122 through an iterative refinement process. The letter generation module 118 facilitates appeal document creation, template management, and the letter recommendation 132 as a part of the feedback loop 136 that monitors changes made during the review process via both the reactive AI module 112 in the form of the letter recommendation 132 and the machine learning module 114 in the form of the editing parameter 134. The letter generation module 118 operates through multiple creation cycles, beginning with generation of the first appeal letter 120 that incorporates the letter recommendation 132, which includes IP-defined definitions, specialized language prompts, and essential medical inputs including standardized definitions of known conditions and medically defined vital readings. The first appeal letter 120 combines the claim data 130, information from the medical database 124, and the letter recommendation 132 to create a comprehensive first appeal letter 120.

It should be appreciated that the letter generation module 118 can use language models to take standard language from input, store it, and enrich it with IP-specific prompts and definitions. The prompts can be interpolated by the machine learning module 114, which creates basic language text blocks that become more complex with iteration and with the use of the IP-related prompts and definitions. The IP-defined definitions include basic definitions of known sicknesses and medically defined vital readings known to the industry, along with the personal experience of the user. The specialized language prompts and IP-defined definitions work together during generation of the first appeal letter 120. As an example, the IP-defined definition can refer to publicly available clinical guidelines, payer standards, insights, and similar evidence-based resources. Over time, as appeal letters are generated and refined, these definitions will be enhanced using feedback and information provided by clinical professionals. The iterative process aims to strengthen the arguments and improve the success rates of appeal letters.

The letter generation module 118 can also be in communication with the machine learning module 114 when creating the second appeal letter 122. By working with the machine learning module 114 to incorporate feedback, the letter generation module 118 can generate the second appeal letter 122 to be tailored to the denied insurance claim 126 and the insurance company based on the editing parameter 134. The letter generation module 118 can use the information gathered by the machine learning module 114, such as if the second appeal letter 122 results in an approved insurance claim and the feedback generated by the feedback loop 136 of the machine learning module 114 in each subsequent iteration of the appeal letter. The communication between the letter generation module 118 and the machine learning module 114 allows the letter generation module 118 to continuously refine and improve the appeal letter generation process based on successful outcomes.

It should be appreciated that the memory 108 of the system 100 can further include or be coupled to a memory 108 (internal or external), which can be coupled to the processor such as the processor 106, for storing information and instructions that can be executed by the processor 106 of the system server 102. The memory 108 can be one or more memories and of any type suitable to the local application environment and can be implemented using any suitable volatile or nonvolatile data storage technology such as a semiconductor-based memory device, a magnetic memory device and system, an optical memory device and system, fixed memory, and removable memory. For example, the memory 108 can consist of any combination of random-access memory (RAM), read only memory (ROM), static storage such as a magnetic or optical disk, hard disk drive (HDD), or any other type of non-transitory machine or computer readable media. The instructions stored in the memory 108 can include program instructions or computer program code that, when executed by the processor 106, enable the system 100 to perform tasks as described herein.

One skilled in the art will also appreciate that the processor 106 can be configured for processing information and executing instructions or operations. The processor 106 can be any type of general or specific purpose processor. In some cases, multiple processors for the processor 106 can be utilized according to other embodiments. In fact, the one or more of the processors 106 can include a general-purpose computer, a special purpose computers, microprocessors, digital signal processors (DSPs), field-programmable gate arrays (FPGAs), application-specific integrated circuits (ASICs), and processors based on a multi-core processor architecture, as examples. In some cases, the processor 106 can be remote from the system 100, such as disposed within a remote platform like the one or more remote platforms of FIG. 1.

The processor 106 can perform functions associated with the operation of system 100 which can include, for example, precoding of antenna gain/phase parameters, encoding and decoding of individual bits forming a communication message, formatting of information, and overall control of the one or more computing platform, including processes related to management of communication resources.

In some embodiments, one or more computing platforms can also include or be coupled to one or more antennas (not shown) for transmitting and receiving signals and/or data to and from the system 100. The one or more antennas can be configured to communicate via, for example, a plurality of radio interfaces that can be coupled to the one or more antennas. The radio interfaces can correspond to a plurality of radio access technologies including one or more of LTE, 5G, WLAN, Bluetooth, near field communication (NFC), radio frequency identifier (RFID), ultrawideband (UWB), and the like. The radio interface can include components, such as filters, converters (for example, digital-to-analog converters and the like), mappers, a Fast Fourier Transform (FFT) module, and the like, to generate symbols for a transmission via one or more downlinks and to receive symbols (for example, via an uplink).

With renewed reference to FIG. 1, and as described hereinabove, the memory 108 stores the plurality of modules, including the data input module 110, the reactive AI module 112, the machine learning module 114, the user interface module 116, and the letter generation module 118, and including the machine-readable instructions, which can be provided as tangible, non-transitory processor executable instructions, for example. The instructions are configured to execute the method 200 of the present disclosure, by the processor 106 or the other processors of the system 100 as detailed herein, and as described hereinbelow and shown in FIG. 1.

Referring now to FIGS. 2A-2C and 3A-3E, aspects of the method 200 for processing a denied insurance claim 126 are shown. The method 200 can include a step 202 of providing the system 100 as described herein. The denied insurance claim 126 can be provided to the data input module 110 of the system 100 in a step 204. In a step 206, the method can include providing the patient history record 128 to the data input module 110 of the system 100. The step 206 of providing the patient history record can include a step 208 of the data input module 110 intaking the patient history record 128 from Electric Health Record software. It should be appreciated that the system 100 can extract data from the HER software through direct database access, API interfaces, or UI software extraction.

The data input module 110 can normalize the denied insurance claim 126 and the patient history record 128 by scrubbing for claim data 130 related to the denied insurance claim 126 and the patient history record 128 in a step 210. It should be noted that the step 210 of normalizing for claim data 130 can include a step 212 of augmenting and enriching the claim data 130 using IP-specific clinical definitions and a step 214 of storing the claim data 130 at either a third or fourth level of normalization in databases.

The method 200 can include a step 216 of suggesting a medical condition related to the denied insurance claim 126. The reactive AI module 112 can make the suggestion based on the medical database 124. By making the suggestion, the system 100 can help to tie together the complete medical reasoning used by the user, which includes the knowledge of family history, medical records, vital signs, and other clinical factors they observed during treatment. By suggesting related conditions, the reactive AI module 112 helps articulate the full clinical context that informed the decision-making of the user but may not be immediately apparent to the payer. The reactive AI module 112 can compare the claim data 130 to the medical database 124, in a step 218, to evaluate multiple criteria and provide context for the first appeal letter 120. The step 218 of comparing the claim data 130 to the medical database 124 can include a step 220 of comparing the claim data 130 to the medical database 124 including evaluating various disease criteria including SOFA, SIRS and SEP-3 criteria, a step 222 of evaluating in-network status, out-of-network status, medical agreed terms limits, and contracted rules and a step 224 of evaluating a coverage limit and policy definition of the insurance company.

In a step 226, the method 200 can include the reactive AI module 112 generating a letter recommendation 132 based on the medical database 124 of the data input module 110. The letter recommendation 132 can contain disease state categorizations, regulations, insurance payor requirements, and industry standards. The letter recommendation 132 can be used by the letter generation module, along with the claim data and medical database information, to create the first appeal letter 120 in a step 228. The letter recommendation 132 helps ensure the first appeal letter 120 incorporates relevant clinical guidelines, state and federal regulations, and clinical best practices needed for a successful appeal. For example, the clinical guideline can be determined by medical associations, such as the American Academy of Family Physicians (AAFP), governmental bodies, such as the Center for Disease Control, and healthcare providers. In a step 230, the letter generation module 118 can employ a language prompt and IP-defined definitions to further tailor the first appeal letter 120 with respect to the denied insurance claim 126.

In a step 232, the first appeal letter 120 can be presented, via the user interface 138 of the user interface module 116, to the user for review and a step 234 of reviewing, by the user, the first appeal letter 120 to determine the editing parameter 134. When reviewing the first appeal letter 120, the user can determine the editing parameter 134 based on specific insurance company requirements. The editing parameter 134 seeks to incorporate approved insurance claim criteria for the particular insurance company being appealed to. As an example, the approved insurance claim criteria can include analyzing appeal letters that have resulted in an approved insurance claim for the editing parameter 134 that resulted in the insurance claim being approved. The editing parameter 134 can be stored by the machine learning module 114 in a step 236. In a step 238, the machine learning module 114 can be updated based on the editing parameter 134 made by the user to the first appeal letter 120. The letter generation module 118 can generate the second appeal letter 122 based on the update to the machine learning module 114 in a step 240.

It should be appreciated that the method steps of reviewing the appeal letter 120, storing the editing parameter 134, updating the machine learning module 114, and generating a subsequent appeal letter 122 can be repeated at the users request as many times required to generate an appeal letter that results in the denied insurance claim 126 being approved by the payor. In this way, the system 100 can create a continuous feedback loop 136 where the system 100 becomes more effective over time at handling appeals for specific insurance companies. It should be appreciated that the system 100 specifically learns which criteria are successful in appealing insurance coverage denials, including aspects related to specific insurance companies, through this editing process. In this way, the method can include a step 242 of generating, by the machine learning module 114, the feedback loop 136 to improve letter generation by varying model weights based on the approved insurance claim. The feedback loop 136 can include a step 246 of scoring, by the machine learning module 114, the second appeal letter 122 based on a predetermined scale to determine a success rate of the second appeal letter. As an example, the predetermined scale can include payer-specific criteria such as in-network/out-of-network status, medical agreed terms limits, contracted rules, coverage limits, and policy definitions specific to each insurance company. Additionally, the system evaluates clinical accuracy by measuring the appeal against established medical criteria. By scoring the second appeal letter 122, the user can determine whether a subsequent appeal letter may need to be generated.

In a step 246, the machine learning module can employ a technique like SHapley Additive explanations (SHAP) or Local Interpretable Model-agnostic Explanations (LIME) during the editing process to provide insight into why specific letter content was generated, allowing for more precise editing and tailoring of the appeal.

To continuously improve the system 100 and provide accurate letter generation, the method can include a step 248 of updating the medical database 124 based on categorization around disease states based on clinical and society guidelines. Additionally, the reactive AI module 112 can be continuously trained through reviewing and selecting relevant aspects for an insurance company as company specific guidelines and regulations are announced in a step 250.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A method for processing a denied insurance claim including modification by a user, the method comprising:
providing a system server having a processor and a memory on which a plurality of modules including tangible, non-transitory, processor executable instructions are stored, the plurality of modules including a data input module, a reactive artificial intelligence (AI) module, a machine learning module, a user interface module, and a letter generation module,
the data input module having a medical database, the data input module in communication with the reactive AI module,
the user interface module permitting interaction with the system server by the user, the user interface module in communication with the letter generation module and the machine learning module, and
the letter generation module configured to generate a first appeal letter and a second appeal letter, the letter generating module configured for permitting for interaction with the first appeal letter and the second appeal letter via the user interface, the letter generation module in communication with the reactive AI module and the machine learning module;
providing the denied insurance claim to the data input module of the system server;
providing a patient history record to the data input module of the system server;
normalizing, by the data input module, the denied insurance claim and the patient history record by scrubbing for claim data related to the denied insurance claim and the patient history record;
comparing, by the reactive AI module, the claim data to the medical database;
generating, by the reactive AI module, a letter recommendation based on the medical database of the data input module;
generating, by the letter generation module, the first appeal letter based on the claim data, the medical database, and the letter recommendation;
presenting, by the user interface, the first appeal letter to the user for review;
reviewing, by the user, the first appeal letter to generate an editing parameter based on an insurance company;
storing, by the machine learning module, the editing parameter generated by the user to the first appeal letter;
updating the machine learning module based on the editing parameter generated by the user to the first appeal letter; and
generating, by the letter generation module, the second appeal letter based on the update to the machine learning module.

2. The method of claim 1, wherein machine learning module includes a feedback loop configured to track if the second appeal letter results in an approved insurance claim, the method further including a step of generating, by the machine learning, a feedback loop to improve letter generation by varying model weights based on the approved insurance claim.

3. The method of claim 1, wherein the step of normalizing the denied insurance claim and the patient history record includes augmenting the denied insurance claim and the patient history record using a clinical definition.

4. The method of claim 1, wherein the step of generating the first appeal letter further includes a step of employing, by the letter generation module, a language prompt and IP-defined definition.

5. The method of claim 1, wherein the patient history record includes a family history and a medical record.

6. The method of claim 1, wherein the step of comparing the claim data against the medical database includes evaluating Sequential Organ Failure Assessment (SOFA), Systemic Inflammatory Response Syndrome (SIRS), and Sepsis-3 (SEP-3) criteria.

7. The method of claim 1, the method includes a step of scoring, by the machine learning module, the second appeal letter based on a predetermined scale to determine a success rate of the second appeal letter.

8. The method of claim 1, wherein the reactive AI module includes a combination of algorithmic training, a large language model, and reactive machine AI.

9. The method of claim 1, wherein the data input module includes a change data capture for making changes to variable weights in algorithmic training.

10. The method of claim 1, further including a step of employing, by the machine learning module, at least one of a SHapley Additive explanation (SHAP) and a Local Interpretable Model-agnostic Explanation (LIME).

11. The method of claim 1, wherein the step of comparing the claim data includes a step of evaluating network status.

12. The method of claim 1, further including a step of updating the medical database based on categorization around disease states based on a clinical guideline.

13. The method of claim 1, further including a step of intaking, by the data input module, the patient history record from Electronic Health Record software.

14. The method of claim 1, wherein the step of comparing the claim data includes a step of evaluating a coverage limit and a policy definition of the insurance company.

15. The method of claim 1, wherein the step of normalizing the data includes a step of storing the data at a third level of normalization.

16. The method of claim 1, including a step of training, by the user, the reactive AI module through reviewing the editing parameter for an insurance company.

17. The method of claim 1, including a step of suggesting, by the reactive AI module, another related medical condition to the denied insurance claim based on the medical database.

18. The method of claim 1, wherein the editing parameter includes incorporating an approved insurance claim criteria for the insurance company.

19. A system for processing a denied insurance claim with a patient history record, comprising:
a system server having a processor and a memory on which a plurality of modules including tangible, non-transitory, processor executable instructions are stored, the plurality of modules including a data input module, a reactive artificial intelligence (AI) module, a machine learning module, a user interface module, and a letter generation module,
the data input module having a medical database, the data input module in communication with the reactive AI module,
the user interface module permitting interaction with the system server by the user, the user interface module in communication with the letter generation module and the machine learning module, and
the letter generation module configured to generate a first appeal letter and a second appeal letter, the letter generating module configured for permitting for interaction with the first appeal letter and the second appeal letter via the user interface, the letter generation module in communication with the reactive AI module and the machine learning module;
the system server configured to:
normalize, by the data input module, the denied insurance claim and the patient history record by scrubbing for claim data related to the denied insurance claim and the patient history record;
compare, by the reactive AI module, the claim data to the medical database;
generate, by the reactive AI module, a letter recommendation based on the medical database of the data input module;
generate, by the letter generation module, the first appeal letter based on the claim data, the medical database, and the letter recommendation;
present, by the user interface, the first appeal letter to the user for review;
store, by the machine learning module, the editing parameter generated by the user to the first appeal letter;
update the machine learning module based on the editing parameter generated by the user to the first appeal letter; and
generate, by the letter generation module, the second appeal letter based on the update to the machine learning module.

20. The system of claim 19, wherein the machine learning module includes a feedback loop configured to track if the second appeal letter results in an approved insurance claim.
